(19) United States
(12) Patent Application Publication
(10) Pub. No.: US 2020/0245701 A1
(43) Pub. Date: Aug. 6, 2020

(54) DEVICE FOR PROTECTIING AGAINST HAIR LOSS WHEN WEARING HEADGEAR

(71) Applicant: Amber Houston, Cordova, TN (US)

(72) Inventor: Amber Houston, Cordova, TN (US)

(21) Appl. No.: 16/267,949

(22) Filed: Feb. 5, 2019

(51) Int. Cl. A41D 20/00; A42C 5/00; A61K 38/17; A61K 9/06

(52) U.S. Cl. CPC A41D 20/00; A42C 5/00; A41D 2400/32; A61K 9/06; A61K 38/1748

(57) ABSTRACT

A protective head band for protecting against hair loss when wearing headgear is provided. The protective head band may be adapted to engage an inner periphery of the headgear. The protective head band may have a gel band providing a plurality of gel nubs adapted to decrease the pulling and sliding friction from the tension to the scalp imposed by the inner periphery.

### BACKGROUND OF THE INVENTION

The present invention relates to hair loss prevention and, more particularly, to a device for protecting against hair loss when wearing headgear.

With the advance of age, many people who wear headgear may experience thinning of their hair along the edges of their scalp where a periphery of the headgear engages, applying tension. For example, for women who wear wigs the underlying head cap portion can promote hear loss where the circumference of the head opening tightly engages the wearer's scalp. In part, this result from the material or fabric that define such head openings—e.g., polyester, nylon, and other synthetic materials. Such synthetic material has a coefficient of friction so that they apply a friction on the scalp sufficient to make the hair become weak and brittle, causing baldness and traction alopecia. On the other hand, cotton and other natural fabrics used for the head opening may apply less tensive force (have a lower coefficient of friction), but tend to absorb moisture, which can also make the affected hairline brittle and thus more susceptible to hair loss.

As can be seen, there is a need for a device for protecting against hair loss when wearing headgear. The device embodied in the present invention provides a plurality of gel nubs spaced apart on a band to be connected along an inner periphery of the head opening of the headgear. The plurality of gel nubs may be incorporated on a band so as to have a plurality of rows of gel nubs. The band and plurality of gel nubs are adapted to hold the headgear in place, while decreasing the pulling and sliding friction from the tension to the scalp imposed by the inner periphery. Furthermore, the gel nub's do not absorb moister, like natural fabrics, and so further protects the hairline from thinning and balding.

### SUMMARY OF THE INVENTION

In one aspect of the present invention, a protective head band for protecting against hair loss when wearing headgear includes the following: a gel band adapted to adorn a hairline of a human wearer; and a plurality of gel nubs spaced apart along a first surface of the gel band so that the plurality of gel nubs generally engages said hairline.

In another aspect of the present invention, protective head band for protecting against hair loss when wearing headgear includes the following: a gel band adapted to adorn a hairline of a human wearer; the gel band having a first surface and an opposing second surface; a plurality of gel nubs spaced apart along the first surface, wherein the plurality of gel nubs form more than one row, wherein the plurality of gel nubs is adapted to align with said hairline; and the second surface connected to an inner periphery of the headgear.

In yet another aspect of the present invention, method of decreasing pulling and sliding friction to the scalp imposed by an inner periphery of worn headgear includes providing the protective head band disclosed above and connecting a second surface to said inner periphery.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is an exploded perspective view of an exemplary embodiment of the present invention;

FIG. 3 is a section view of an exemplary embodiment of the present invention, taken along line 3-3 in FIG. 1;

FIG. 4 is a detail section view of an exemplary embodiment of the present invention; and

FIG. 5 is a section detail view of an exemplary embodiment of the present invention, illustrating a gel nub.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides protective head band for protecting against hair loss when wearing headgear. The protective head band may be adapted to engage an inner periphery of the headgear. The protective head band may have a gel band providing a plurality of gel nubs adapted to decrease the pulling and sliding friction from the tension to the scalp imposed by the inner periphery.

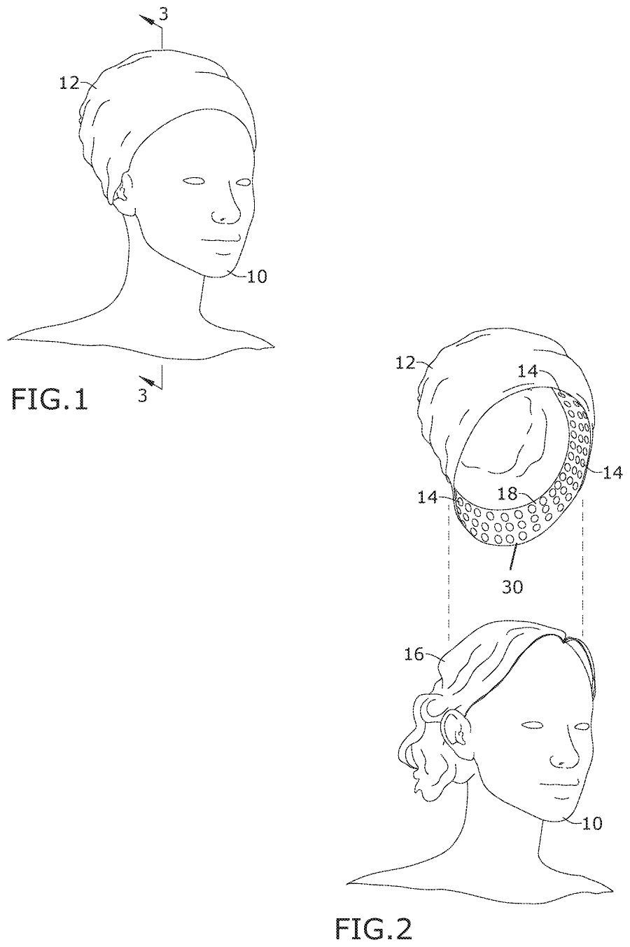

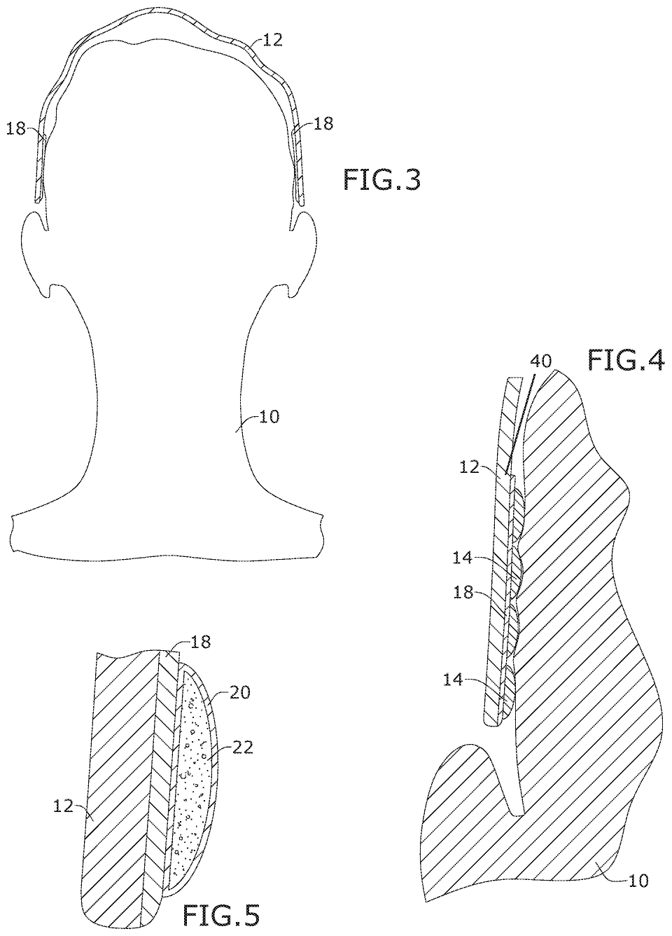

Referring to FIGS. 1 through 5, the present invention may include a protective hair band 30 dimensioned and adapted to connect to an inner periphery 40 of any headgear 12. The protective hair band 30 protects the wearer's 10 hair(line) 16 from hair loss due to the wearing the headgear 12.

It should first be understood that the headgear 12 can be any article worn on the head of a human wearer. Including, but not limited to a cap, headwear, helmet, wig, hat, etc.

The protective hair band 30 includes a gel band 18 that may be an endless loop, but alternative be less than an endless loop and so less than coextensive with said inner periphery 40. The protective hair band 30 may be joined to the inner periphery 40 by various conventional methods, such as adhesives, threading, hook and loop fasteners, and the like. Alternatively, the protective hair band 30 may be modular and separable from the headgear 12.

Along the surface of the protective hair band 30 are disposed a plurality of gel nubs 14. Each gel nub 14 may be a capsule 20 housing liquid or viscous gel 22, such as keratin. Each gel nub 14 may be approximately a quarter to one inch in diameter.

In certain embodiments, the gel band 18 too may be filled with the gel 22. In some of these cases, the gel band 18 and capsules 20 may be fluidly connected. In many embodiments, the plurality of gel nubs 14 form a plurality of rows as they are spaced along the gel band 18. In the embodiments where the plurality of gel nubs 14 (and/or the gel band 18) are not coextensive with the inner periphery 40 the plurality of gel nubs 14 may be strategically placed so as to be adapted to align with the temples, hair line, or nape of the neck of the wearer (directly above the hair line).

A method of using the present invention may include the following. The protective hair band 30 disclosed above may be provided. To prevent alopecia, hair thinning, or breakage in any areas, either on the sides or in the nape of the neck of the head, a user may apply the protective hair band 30 to the inner periphery 40 of the selected headgear 23 cap. The plurality of the gel nubs 14 on the gel band 18 will act as the stopping agent from the prevention of friction (which causes hair loss). It is important to obtain the appropriate head size of the gel caps for better results.

It should be understood, of course, that the foregoing relates to exemplary embodiments of the invention and that modifications may be made without departing from the spirit and scope of the invention as set forth in the following claims.

What is claimed is:

1. A protective head band for protecting against hair loss when wearing headgear, comprising:
a gel band adapted to adorn a hairline of a human wearer; and
a plurality of gel nubs spaced apart along a first surface of the gel band so that the plurality of gel nubs generally engages said hairline.

2. The protective head band of claim 1, wherein the plurality of gel nubs form more than one row.

3. The protective head band of claim 1, wherein the plurality of gel nubs is coextensive with said hairline.

4. The protective head band of claim 1, wherein the plurality of gel nubs is adapted to align with said hair line.

5. The protective head band of claim 1, wherein the plurality of gel nubs provides keratin.

6. The protective head band of claim 1, wherein the gel band is filled with gel.

7. The protective head band of claim 6, wherein the gel band and the plurality of gel nubs are fluidly connected.

8. The protective head band of claim 6, further provide a second surface to the gel band; the second surface opposing the first surface; and the second surface connected along an inner periphery of the headgear.

9. A protective head band for protecting against hair loss when wearing headgear, comprising:
a gel band adapted to adorn a hairline of a human wearer;
the gel band having a first surface and an opposing second surface;
a plurality of gel nubs spaced apart along the first surface, wherein the plurality of gel nubs form more than one row, wherein the plurality of gel nubs is adapted to align with said hairline; and
the second surface connected to an inner periphery of the headgear.

10. The protective head band of claim 9, wherein the gel band is filled with gel.

11. The protective head band of claim 10, wherein the gel band and the plurality of gel nubs are fluidly connected.

12. A method of decreasing pulling and sliding friction to the scalp imposed by an inner periphery of worn headgear, comprising:
providing the protective head band of claim 1; and
connecting a second surface to said inner periphery.
